(19) United States
(12) Patent Application Publication
(10) Pub. No.: US 2018/0271364 A1
(43) Pub. Date: Sep. 27, 2018

(54) METHOD AND APPARATUS FOR DETECTING OCULAR MOVEMENT DISORDERS

(71) Applicants: OSAKA UNIVERSITY, Osaka (JP); University of Kansas, Lawrence, KS (US); Augusta University Research Institute, Inc., Augusta, GA (US)

(72) Inventors: Jason Edward Orlosky, Minoh City (JP); Hannes Devos, Overland Park, KS (US); John Christopher Morgan, Martinez, GA (US)

(21) Appl. No.: 15/934,479

(22) Filed: Mar. 23, 2018

Related U.S. Application Data
(60) Provisional application No. 62/476,530, filed on Mar. 24, 2017.
(60) Provisional application No. 62/542,908, filed on Aug. 9, 2017.

(51) Int. Cl. A61B 3/113; G06F 3/01; A61B 3/00; A61B 3/14; A61B 5/00; A61B 3/11

(52) U.S. Cl. CPC A61B 3/113; G06F 3/013; A61B 3/112; A61B 3/14; A61B 5/4082; A61B 3/0058

(57) ABSTRACT

A system for identifying abnormal eye movements includes a near-eye display (NED), an eye-tracking camera, a frame supporting the NED and the eye-tracking camera, and a processor in data communication with the NED, the eye-tracking camera, and a computer readable medium. The computer readable medium has instructions thereon. When executed by the processor, the instructions cause the processor to provide a target on the NED to a user's eye and change the target or move the target to a plurality of locations in three dimensions on the NED according to one or more tasks of a task module. The processor further records positional information and pupil information of the user's eye during the one or more tasks of the task module and compares the positional information to at least one threshold value of an abnormality identification algorithm.

### CROSS-REFERENCE TO RELATED APPLICATIONS

This application claims the benefit of and priority to U.S. Provisional Patent Application No. 62/476,530, filed on Mar. 24, 2017, and U.S. Provisional Patent Application No. 62/542,908, filed on Aug. 9, 2017, which are hereby incorporated by reference in their entireties.

### BACKGROUND OF THE DISCLOSURE

Parkinson's disease (PD) is the second most prevalent neurodegenerative condition. Hallmark characteristics of the disease are shaking (tremor), slowness (bradykinesia), muscle stiffness (rigidity), postural instability, and gait disorders. Many other neurodegenerative conditions present with a similar clinical picture, but are not considered PD. These are classified as Parkinson plus disorders and also fall under the umbrella term Parkinsonism. Accurate diagnosis of these different forms of Parkinsonism is critical to deliver the best medical treatment. Early attempts to measure motor skills via computers date back to 1986, and different techniques have been developed over the years to assist with differential diagnosis of these conditions, including the detection of eye movement abnormalities. In Parkinsonism, several brain areas from the brain stem to higher cortical areas that are crucial for saccade generation and smooth pursuit may be disturbed.

In the differential diagnosis of parkinsonism and their related eye movement disorders, clinicians currently focus on the execution of saccadic movements and smooth pursuit. The role of saccades is to direct the fovea of the eye to an object of interest, and smooth pursuit eye movements are responsible for keeping the fovea on the object. Smooth pursuits are defined as conjugate eye movements that track a moving object in order to maintain fixation. There are different forms of saccadic movements, and visually guided prosaccades and antisaccades are of particular interest in the differential diagnosis of parkinsonian disorders. Prosaccades are reflexive saccades towards the object of interest. Antisaccades are saccades to the opposite side of the object of interest. The initiation of these saccades may be delayed (increased saccadic latency), the saccades may be slower (bradykinesia) than anticipated, or may not shift exactly on the target (hypometria) in patients with parkinsonism. Unique findings as well as subtle differences in oculomotor abnormalities within apparently similar conditions may be elicited if the patient is examined carefully. For example, patients with progressive supranuclear palsy, a type of parkinsonism, present with vertical gaze palsy, which is less likely to be present in other types of parkinsonism. Some of these eye movement abnormalities are more subtle and hard to detect with the naked eye.

Supranuclear Palsy, Multiple System Atrophy, and Cortico-basal Degeneration, often present with a similar clinical picture. These disorders are therefore classified as parkinsonism. Although neuroimaging techniques may help differentiate between these neurodegenerative conditions, the diagnosis is conventionally made in the clinic based on visible clinical signs and symptoms. However, patients often wait for years for a correct diagnosis. Improving the speed, consistency, and portability of methods for diagnosis is critical for delivering the right treatment and medication at the right time.

An intrinsic part of the skill set of neurologists is the identification of eye movement abnormalities, which are common in Parkinsonism and can sometimes be elicited by physical clinical tests. An example of the most basic of these is to have a patient fixate on the physician's finger and to watch the patient's eye movements for abnormalities during the task. Other tools such as eye tracking interfaces are available to give the physician a better view of the patient's eye movements. However, even with current eye tracking interfaces, physicians are still prone to error. Misdiagnosis is also still common, especially at initial stages of certain diseases, including PD. A physician's finger movements may not be adequate in evoking a response, and minute abnormalities are not always visible due to limited resolution or perception. Both physician and patient must also be physically present in the same area, and access to health care is often difficult in remote areas.

### SUMMARY

In some embodiments, a system for identifying abnormal eye movements includes a near-eye display (NED), an eye-tracking camera, a frame supporting the NED and the eye-tracking camera, and a processor in data communication with the NED, the eye-tracking camera, and a computer readable medium. The computer readable medium has instructions thereon. When executed by the processor, the instructions cause the processor to provide a target on the NED to a user's eye and change the target or move the target to a plurality of locations on the NED according to one or more tasks of a task module. The processor further records positional information of the user's eye during the one or more tasks of the task module and compares the positional information to at least one threshold value of an abnormality identification algorithm.

In other embodiments, a method of identifying abnormal eye movements in a patient includes providing a target on the NED to a user's eye and moving the target or move the target to a plurality of locations on the NED according to one or more tasks of a task module. The method further includes recording positional information of the user's eye during the one or more tasks of the task module and comparing the positional information to at least one threshold value of an abnormality identification algorithm.

In yet other embodiments, a method abnormal eye movements in a patient includes providing a target on the NED to a user's eye and moving the target to a plurality of locations on the NED according to at least a stationary task of a task module. The method further includes recording positional information of the user's eye during the stationary task of the task module and identifying at a plurality of oscillation/ocular tremors between 4 Hz and 7 Hz in the positional information.

This summary is provided to introduce a selection of concepts that are further described below in the detailed description. This summary is not intended to identify key or essential features of the claimed subject matter, nor is it intended to be used as an aid in limiting the scope of the claimed subject matter.

Additional features and advantages of embodiments of the disclosure will be set forth in the description which follows, and in part will be obvious from the description, or may be learned by the practice of such embodiments. The features and advantages of such embodiments may be realized and obtained by means of the instruments and combinations particularly pointed out in the appended claims. These and other features will become more fully apparent from the following description and appended claims, or may be learned by the practice of such embodiments as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 is a schematic view of an embodiment of a system for detecting ocular movement abnormalities, according to at least one embodiment of the present disclosure;

FIG. 2 is a system flowchart illustrating an embodiment of a system for stimulating, detecting, segmenting, and visualizing ocular movement abnormalities, according to at least one embodiment of the present disclosure;

FIG. 3 is a flowchart illustrating an embodiment of a task module of a diagnostic system, according to at least one embodiment of the present disclosure;

FIG. 4 is a flowchart illustrating an embodiment of an abnormality detection algorithms module of a diagnostic system, according to at least one embodiment of the present disclosure;

FIG. 5 is a graph illustrating example data of ocular oscillation detected with an embodiment of a diagnostic system, according to at least one embodiment of the present disclosure;

FIG. 6 is a graph illustrating example data of abnormal saccade intrusions detected with an embodiment of a diagnostic system, according to at least one embodiment of the present disclosure;

FIG. 7 is a flowchart illustrating an embodiment of a visualization module of a diagnostic system, according to at least one embodiment of the present disclosure; and

FIG. 8 is a flowchart illustrating an embodiment of a machine learning module of a diagnostic system, according to at least one embodiment of the present disclosure.

### DETAILED DESCRIPTION

This disclosure generally relates to devices, systems, and methods for identifying eye movement and pupil abnormalities. More particularly, the present disclosure relates to stimulating and/or detecting ocular behavior correlated to eye movement and pupil abnormalities. For example, certain tremors and/or movement patterns of the eye may indicate early stages of ocular movement abnormalities such as Parkinson's disease (PD). In some patients, the ocular behaviors may be visible to a medical professional, but in other patients, the ocular tremors may be very slight (e.g., sub-millimeter) and abnormal patterns may be difficult to identify, even for experienced professionals. Furthermore, current diagnosis of ocular behaviors linked to eye movement and pupil abnormalities requires a specialist present for the diagnosis.

In some embodiments, a system for identifying abnormal ocular behavior may include a near-eye display (NED) and an eye-tracking system. The eye-tracking system may be integrated into the near-eye display, limiting or preventing the need for calibration of the system for different users. The system may allow for the display of a variety of stimuli and the detection of sub-pixel movement of the user's eye for diagnostic purposes. For example, the system may image the user's eye to detect and track at least a centroid of the user's pupil. In other examples, the system may detect the size and/or rate of change in size of the user's pupil.

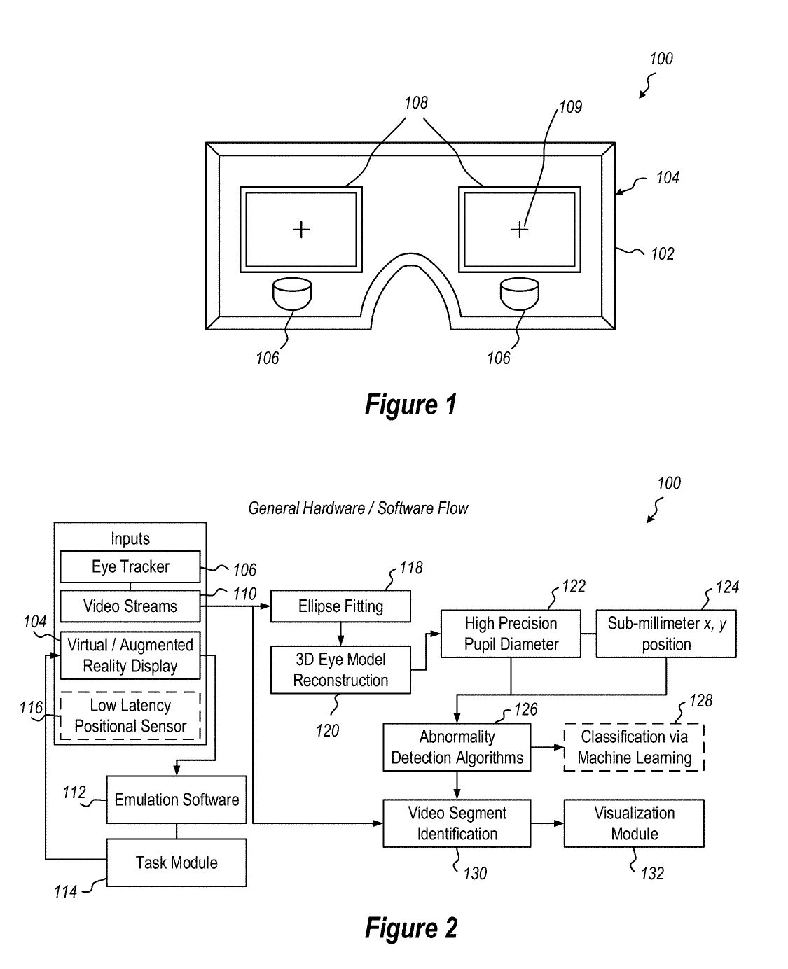

FIG. 1 illustrates an interior of an embodiment of a diagnostic system 100, according to the present disclosure. The diagnostic system 100 may include a frame 102 that supports a NED 104 and an eye-tracking camera 106. The NED 104 may include a plurality of display screens 108 (e.g., one display screen for each eye), that may be occlusive, transparent, or semi-transparent. In another example, the NED 104 may include a single display screen that is positioned in front of both eyes and displays images to both eyes (e.g., a split-screen display), such as an implementation of augmented reality, mixed reality, or virtual reality, or some combination thereof.

In some embodiments, the display screens 108 may be rectangular, as shown in FIG. 1. In other embodiments, the display screens 108 may be circular, elliptical, or otherwise rounded. In yet other embodiments, the display screens may be at least partially rounded, at least partially polygonal, regular polygonal, irregularly shaped, or combinations thereof.

The diagnostic system 100 may include a plurality of eye-tracking cameras 106. For example, the diagnostic system 100 may have one eye-tracking camera 106 for each eye of the user. The eye-tracking cameras 106 may image the user's eye and measure movement of the user's eye.

The diagnostic system 100 may present a target 109 to a user on the NED 104. The target 109 may move on the NED 104 in different directions and/or at different rates to stimulate particular behaviors that may indicate the presence of one or more ocular movement abnormalities. For example, the target 109 may be held stationary on the NED 104 and the eye-tracking cameras 106 may monitor the position of the user's pupil. Small oscillatory tremors of the pupil or saccadic intrusions may indicate Parkinsonism.

FIG. 2 is a system diagram that illustrates the processes of the diagnostic system 100 used to present a target to a user in a series of tasks, to track the user's eye in response to the series of tasks, and to detect and classify abnormalities in the eye movement.

The system 100 includes the NED 104 of the virtual/augmented reality display and the eye-tracking camera 106. The eye-tracking camera 106 generates a video stream 110 for each of the eye-tracking cameras 106. The video stream 110 includes the movement of the user's eye in the field of view of the camera. The detection and tracking of the user's eye within the video stream 110 may be at least partially dependent upon a resolution and/or a framerate of the video stream 110. In some embodiments, the video stream 110 may have a resolution of at least 480×480 pixels. For example, the video stream 110 may have a resolution of 640×480 pixels. In other embodiments, the video stream 110 may have a resolution of greater than 480×480 pixels. For example, the video stream 110 may have a vertical and horizontal resolution of at least 640 pixels, at least 720 pixels, at least 900 pixels, at least 1080 pixels, or greater resolution.

In some embodiments, the video stream 110 may have a framerate of at least 30 frames per second (FPS). In other embodiments, the video stream 110 may have a framerate of at least 60 FPS. In yet other embodiments, the video stream 110 may have a framerate of at least 120 FPS. Higher framerates may allow for provide more precise rate of change calculations in eye position and pupil diameter.

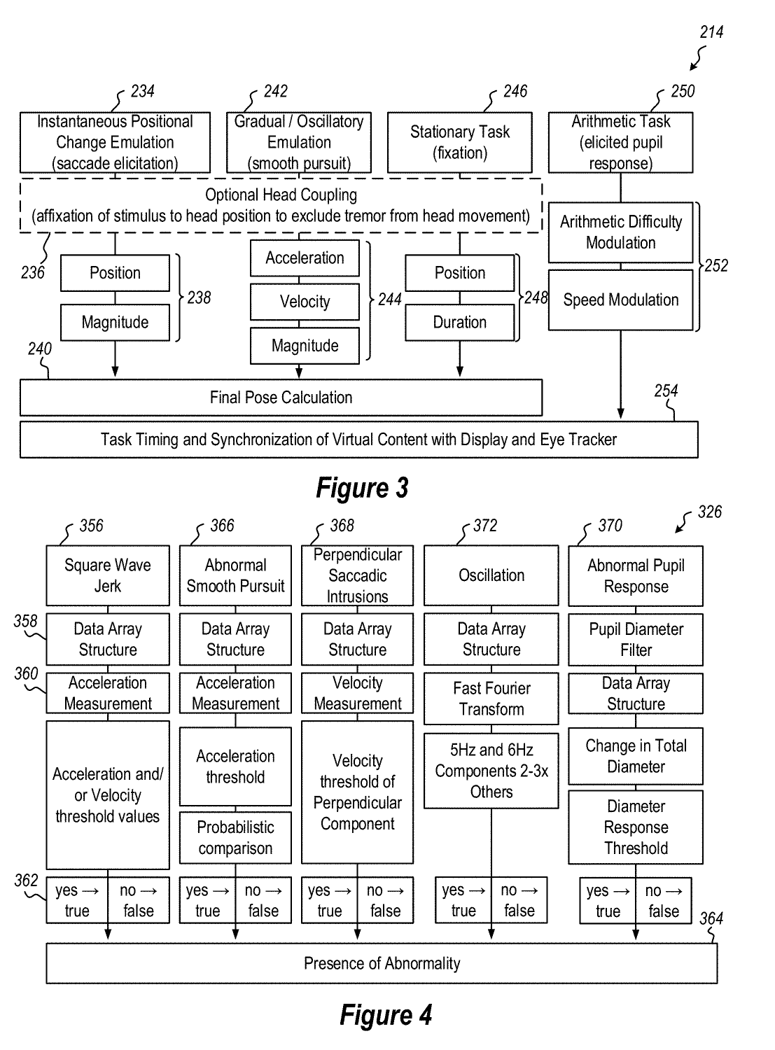

The NED 104 may display images generated by an emulation software 112 and a task module 114. The emulation software 112 may be a custom or existing emulation software 112, such as UNITY. The task module 114 may instruct the NED 104 to display a target 109 in a series of positions and/or patterns that may be used for automatic calibration and may stimulate a particular response or behavior. The task module 114 and particular examples of tasks are described in relation to FIG. 3. Referring again to FIG. 2, a low-latency positional sensor 116 may provide additional information regarding the physical position of the diagnostic system 100 as the diagnostic system 100 and the user may move during diagnostic procedures. For example, the system 100 may detect tremors of the head and neck and differentiate movement of the eye from movement of the head and the system 100.

The video stream 110 may be provided to a microprocessor for one or more processes to be applied to the video stream 110. In some embodiments, the microprocessor may be contained within a head-mounted device, such as the frame 102 depicted in FIG. 1. In other embodiments, the microprocessor may be located externally to the frame, such as in a desktop computer to which the eye-tracking camera 106 is in data communication. In yet other embodiments, the microprocessor may be located remotely to the frame 102, such as over an internet connection to allow the NED 104 and eye-tracking cameras 106 to be operated remotely to diagnose a user without an operator present.

An ellipse fitting procedure 118 may be performed on the video streams 110 to identify the user's pupils. Different ellipse fitting procedures 118 known in the field may be used to identify the user's pupil. For example, an ellipse fitting procedure 118 may include detecting a portion of the pupil region and setting it as the region of interest (ROI) for further processing. The pupil detector is a low contrast area-detection algorithm that identifies dark regions with minimal texture in dynamic scenes. The pupil region in most eye tracking applications is the darkest, most uniform region in the image. Therefore, this identification works very well for finding the pupil when parameterized.

In one example, an algorithm computes a darkness factor for a square region representing the minimum pupil size possible in any video. Each n^{th} pixel in both x and y directions in the image is sampled and added to a total darkness factor for the sampling region. In other words, every n^{th} pixel is sampled in both x and y directions when searching and also when computing darkness. The benefit of this strategy is that it can deal more effectively with shadows and eye lashes since the space between lashes often contains bright pixels.

Upon identification of the pupil, the identified region may be expanded by adding like pixels until the region approaches the boundaries of the user's pupil. Due to the known geometric relationship of the eye-tracking cameras 106 relative to the user's eye, the shape of the ellipse fit to the pupil allows calculation of the orientation of the eye and three-dimensional (3D) eye model reconstruction 120.

After 3D eye model reconstruction 120, the model may be used to measure pupil information such as the pupil diameter 122 and a sub-millimeter pupil position 124 of the pupil. The position of the pupil is measured at a sub-pixel and/or sub-millimeter scale to allow for the detection of microsaccades and ocular tremors that may not be readily visible to an unassisted physician. For example, an ocular tremor may manifest as a 0.27-degree movement of the patient's eye. The 0.27-degree movement is nearly imperceptible to an unassisted physician, but detectable with near-eye video capture in the video streams 110. In some embodiments, the video streams 110 may undergo one or more video enhancements to better visualize movements.

In some embodiments, the eye-tracking camera may have an angular resolution less than 0.1 degree. In other embodiments, the eye-tracking camera may have an angular resolution less than 0.09 degrees. In yet other embodiments, the eye-tracking camera may have an angular resolution less than 0.08 degrees. In at least one embodiment, the eye-tracking camera may have an angular resolution that is about 0.84 degrees.

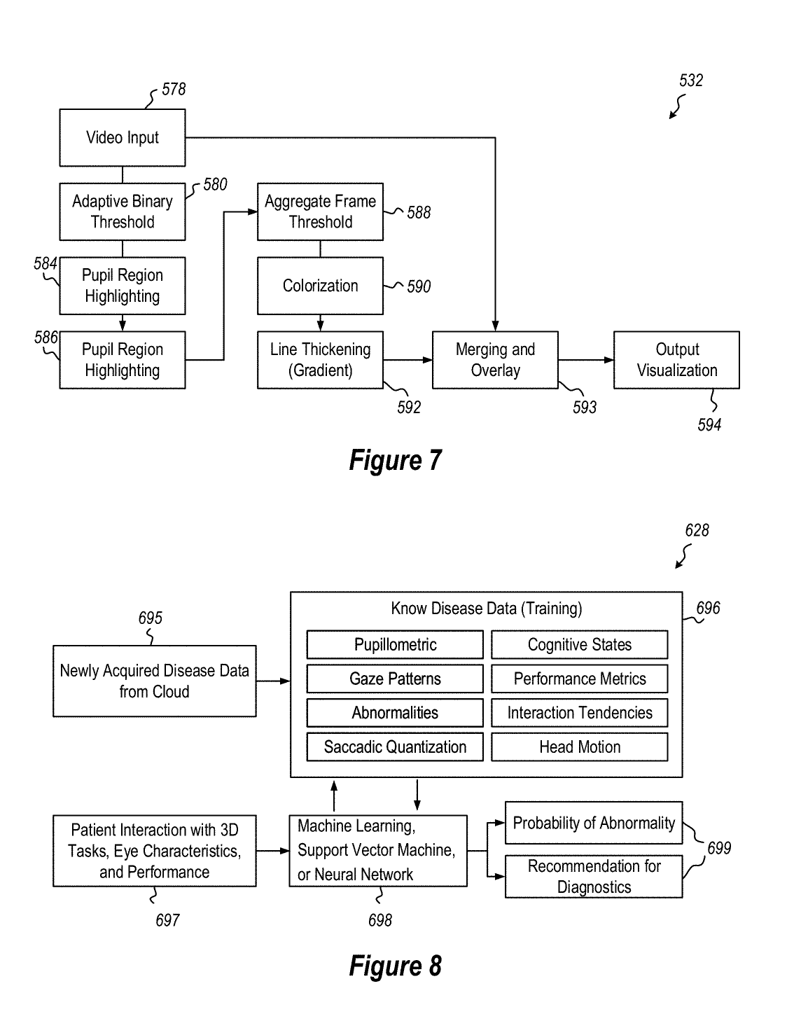

The high precision pupil diameter 122 and sub-millimeter pupil position 124 (including sub-millimeter movement) data may subsequently be provided to one or more abnormality detection algorithms 126 that may identify diagnostic information for classification via machine learning 128 and for video segment identification 130. After video segment identification 130, the segments may be provided to a visualization module 132. Embodiments of abnormality detection algorithms 126 are described in more detail in relation to FIG. 4, embodiments of classification via machine learning 128 are described in more detail in relation to FIG. 8, and embodiments of visualization modules 132 are describe in more detail in relation to FIG. 7.

FIG. 3 is a flow chart illustrating an embodiment of a task module 214, according to the present disclosure. The task module 214 may include tasks configured to elicit eye movements and pupil changes and/or positioned that may be used to detect abnormalities in eye movement and pupils and/or control. The task module 214 may include instantaneous position change emulation 234 to test rapid positional changes in the eye (saccades). The instantaneous position change may move a target (such as target 109 of FIG. 1) to various locations on the NED. As the user's eye moves to track, gaze at, or interact with the target, the system may track the user's pupil through the eye-tracking cameras.

In some embodiments, the system may monitor the movement of the user's head and/or of the system itself, and the system may subtract out the movement of the user's head and/or the system. For example, a patient with Parkinsonism may exhibit tremors, not only in eye movement, but in other muscular movements, which may contribute to relative movement of the eye and the eye-tracking cameras. The optional head coupling 236 may subtract out such movement to isolate the ocular movement and/or rotation.

During the instantaneous position change emulation 234, the task module 214 may vary one or more instantaneous position change variables 238 of the instantaneous position change emulation 234 to measure the user's response to a range of stimuli. For example, the instantaneous position change variables 238 may include the position and/or magnitude of the positional change. In some embodiments, the positional change may have a minimum magnitude relative to the dimensions of the NED. For example, the magnitude of the positional change may be at least 10% of the width and/or height of the NED. In other examples, the magnitude of the positional change may be at least 20% of the width and/or height of the NED. In yet other examples, the magnitude of the positional change may be at least 30% of the width and/or height of the NED. In other words, in a NED with a 640×480 pixel resolution, a positional change with a magnitude of 20% of the width may be a 128-pixel movement in the lateral direction. In some embodiments, the target may move at least 8 pixels on the NED. In other embodiments, the target may move at least 16 pixels on the NED. In yet other embodiments, the target may move at least 24 pixels on the NED.

In some embodiments, the magnitude of the positional change may be relative to an angular position of the target relative to a center point of the user's field of view. For example, the positional change may be within 30 degrees of the center point of the user's field of view on the NED. In other examples, the target may be located greater than 30 degrees from the center point of the user's field of view on the NED. In yet another example, the target may move about 180 degrees across the user's field of view to evaluate peripheral vision.

In some embodiments, the positional change may have a positional change in any direction. For example, a first iteration of the instantaneous position change emulation 234 may include a 100-pixel movement in a lateral direction, a second iteration of the instantaneous position change emulation 234 may include a 100-pixel movement in a vertical direction, and a third iteration of the instantaneous position change emulation 234 may include a 100-pixel movement in an anterior or posterior direction (towards or away) using stereoscopic manipulation. In further iterations, the direction of the positional change of the instantaneous position change emulation 234 may be any direction between lateral, vertical, anterior or posterior, such as a 45-degree angle, a 60-degree angle, a 20-degree angle, or any other angle desired to elicit a saccade or accommodative response. In at least one example, a procession of target locations displayed to a user may include the following (x, y, z) positions, where the x-position, y-position, and z-position are each in apparent meters in the virtual environment: (−1, 3, 5), (1, 3, 5), (3, 0, 5), (0, 3, 5), (0, −2, 5). Upon moving the target through a sequence of target positions, the instantaneous position change emulation 234 may include a final pose calculation 240.

The task module 214 may include a smooth pursuit simulation or a gradual/oscillatory emulation 242. The gradual/oscillatory emulation 242 may move the target in the NED in a continuous manner without sudden displacements of the target. For example, (after the optional head coupling 236) the gradual/oscillatory emulation 242 task may include moving the target with different gradual/oscillatory variables 244 including changes in acceleration, velocity, amplitude, transparency, stereoscopic depth, focal depth, or combinations thereof.

In some embodiments, the gradual/oscillatory emulation 242 may include moving the target with a constant velocity over a particular distance. In other embodiments, the movement of the target may have a varying velocity (i.e., acceleration) which may be constant or may, itself, vary over the duration of the target movement. For example, the target may begin movement by increasing in velocity to a maximum velocity (with a constant or varying positive acceleration), and then decreasing to a stationary location and/or reversing direction (with a constant or varying negative acceleration. In some embodiments, the target may move along a line, while in other embodiments, the target may move in at least two axes, allowing for movement along a first line and a second line oriented at an angle to the first line, or allowing for movement along a curved path, such as moving the target in a circle, ellipse, sphere, ellipsoid or another curved segment. Upon moving the target through a sequence of target movements, the instantaneous position change emulation 234 may include a final pose calculation 240.

In other embodiments, the task module 214 may include a stationary task 246 to test fixation of the user's eye. The stationary task 246 may include (after optional head coupling 236) displaying the target in a sequence of locations and holding the target fixed at each location. For example, the target display may vary stationary variables 248 including the position of the target and duration of time for which the target is displayed. In some embodiments, the target may be displayed for a duration in a range having an upper value, a lower value, or upper and lower values including any of 1 second, 2 seconds, 3 seconds, 4 seconds, 5 seconds, 6 seconds, 7 seconds, 8 seconds, 9 seconds, 10 seconds, or any values therebetween. For example, the target may be displayed for a duration greater than 1 second. In other examples, the target may be displayed for a duration less than 10 seconds. In yet other examples, the target may be displayed for a duration between 1 second and 10 seconds. In further examples, the target may be displayed for a duration between 3 seconds and 8 seconds. In at least one example, the target may be displayed for a duration of 5 seconds.

In some embodiments, the positional change may have a positional change in any direction. For example, a first iteration of the stationary task 246 may include a 100-pixel displacement from the center of the NED in a lateral direction, and a second iteration of the stationary task 246 may include a 100-pixel displacement from the center of the NED in a vertical direction. In further iterations, the displacement from the center of the NED of the target of the stationary task 246 may be any direction between lateral, vertical, anterior, and posterior, such as a 45-degree angle, a 60-degree angle, a 20-degree angle, or any other angle desired. In at least one example, a procession of target locations displayed to a user may include the following (x, y, z) positions, where the x-position, y-position, and z-position are each in apparent meters in the three dimensional virtual environment: (−1, 3, 5), (1, 3, 5), (3, 0, 5), (0, 3, 5), (0, −2, 5). Upon moving the target through a sequence of target positions, the stationary task 246 may include a final pose calculation 240.

In yet other embodiments, a task module 214 may include an arithmetic or mathematics based task 250 to elicit a pupil response. For example, the arithmetic task 250 may vary one or more arithmetic variables 252 to elicit differing pupil responses. In an example, the arithmetic task 250 may include addition of random numbers shown on the NED. The position of the random numbers may vary during the arithmetic task 250. The arithmetic task 250 may include arithmetic problems of varying or increasing difficulty, such as larger or more complex arithmetic problems (e.g., 2+2, in comparison to 1978+377). The arithmetic task 250 may further include varying or increasing the speed at which successive numbers are displayed.

For example, a user may be instructed to add the numbers displayed and say the sum aloud (continuing to add new numbers to the previous sum). For example, if the sequence is 1, 5, 9, and 2, the participant would say 1, 6, 15, and 17. The speed at which numbers appear and the range of numbers to add may increase over the course of a number of trials, for example, with the first trial showing four numbers between 1 and 5 at 5 seconds each, and the last trial showing 10 numbers between 1 and 20 at 2 seconds each. The range of numbers may increase by 5 and the speed at which numbers changed may increase by 1 second for every subsequent trial.

The eye-tracking camera may be tracking and capturing the position and diameter of the user's pupil during each of the tasks in the task module 214 and correlating the task timing and synchronization of the virtual content with the display and eye tracking 254 for diagnostics.

FIG. 4 illustrates embodiments of at least some of the abnormality detection algorithms 326 of a system, according to the present disclosure. In some embodiments, the abnormality detection algorithms 326 may include a square wave jerk algorithm 356. Increased square wave jerks (SWJs), or small, conjugated saccades which take the eyes away from a fixation position and return to the origin, are often a sign of neurodegeneration, especially for diseases like Progressive Supranuclear Palsy and other types of parkinsonism. The square wave jerk algorithm 356 may include accessing a data array structure 358 and evaluate an acceleration measurement 360 of the pupil movement.

During the stationary task, for example, a number of SWJs across a number of patients, several of which were confirmed in follow-up experiments with physicians. The square wave jerk algorithm 356 allows a quantification of the SWJs for each video stream. For fixation tasks, average SWJ frequency between PD and control was 15.8 and 8.0, respectively. The square wave jerk algorithm 356 may compare the measured pupil center displacement against a threshold to test for a presence of the SWJ.

For example, the velocity of the pupil movement and the amplitude of the pupil movement may each have lower thresholds. In some embodiments, the velocity threshold may be about 200 degrees/s and the amplitude threshold may be about 15 pixels. In other embodiments, the velocity may be in a range from 50 degrees per second to 500 degrees per second. For example, the pupil movement may have a velocity in the eye-tracking camera video stream of about 150 pixels per second. The square wave jerk algorithm 356 may compare these values against the velocity threshold and the amplitude threshold in a minimum number of frames. For example, the system may require the thresholds to be met and/or exceeded in at least five out of eight frames.

If the measured pupil movement satisfies both or all thresholds and is followed by a return to center (e.g., the original position before the saccadic intrusion), a Boolean test 362 may return a message to the user and/or to the system of the presence of the abnormality 364.

In some embodiments, the abnormality detection algorithms 326 may include an abnormal smooth pursuit algorithm 366. The abnormal smooth pursuit algorithm 366 checks the data measured from the video streams for abrupt movements of the pupil center during what should be continuous movement. For example, the patient should follow the gradual/oscillatory emulation task described in relation to FIG. 2 with a continuous, gradual movement of the eye. Discontinuous or abrupt movements may be an indicator for one or more neuro-degenerative disorders.

The abnormal smooth pursuit algorithm 366 may access a data array structure 358 and evaluate an acceleration measurement 360 of the pupil movement. In some embodiments, the abnormal smooth pursuit algorithm 366 may compare any abrupt movements during the smooth pursuit to a threshold value. Abrupt movements with acceleration greater than the threshold value may be abnormal saccadic intrusions. In other embodiments, the threshold value may be a velocity threshold. For example, the system may determine that any saccade that exhibits a velocity more than 5, 10, or 15, pixels per second greater than the target movement, may be a potential abnormal saccadic intrusion.

Any movements that exceed the threshold value may be then evaluated with a probabilistic comparison to known examples of saccadic intrusions. For example, the presence of movement meeting and/or exceeding the threshold values in a greater number of measured frames may increase the probability of a positive identification. As described herein, the system may measure movement values that exceed the threshold values in five out of eight frames. In another example, the system may measure movement values that exceed the threshold values in eight out of eight frames, and report an accordingly higher probability of detection.

If the measured pupil movement satisfies both or all thresholds of the abnormal smooth pursuit algorithm 366, a probability or Boolean test 362 may return a message to the user and/or to the system of the presence of an ocular movement abnormality 364, or a visualization applied to the recorded video 594 may be returned to the user for further analysis.

In other embodiments, the abnormality detection algorithms 326 may include a perpendicular saccadic intrusions algorithm 368. Perpendicular saccadic intrusions, e.g., horizontal intrusions in vertical smooth pursuit, have not yet been observed in parkinsonism to our knowledge, though it was apparent in several videos. A perpendicular saccadic intrusions algorithm 368 may specifically evaluate the measured pupil movement for such perpendicular saccadic intrusions by accessing the data array structure and evaluating the instantaneous velocity measurements of the video streams. The perpendicular saccadic intrusions algorithm 368 may include one or more velocity thresholds with vector perpendicular to the anticipated direction. For example, velocity data that is collected and correlated to the gradual/oscillatory emulation task for a control (i.e., healthy) subject, the velocity should be similar or the same to the velocity of the gradual/oscillatory emulation task. When the gradual/oscillatory emulation task has a constant velocity, the velocity measurements evaluated by the perpendicular saccadic intrusions algorithm 368 should be constant both in vector magnitude and direction. If the direction of the vector changes to perpendicular to the vector direction of the target from the gradual/oscillatory emulation task, a perpendicular saccadic intrusion may be present.

In some embodiments, the velocity threshold may have both a magnitude and direction component. For example, the velocity threshold may be at least 30 degrees per second of rotation in a perpendicular direction. While the actual movement of the pupil may be in a non-perpendicular direction, the velocity threshold may consider a perpendicular component of the velocity vector to evaluate the presence of a perpendicular saccadic intrusions. For example, the velocity threshold may be movement of greater than five pixels per frame in a direction perpendicular to the direction of elicited smooth pursuit, for five frames out of any eight consecutive frames.

In yet other embodiments, the abnormality detection algorithms 326 may include an abnormal pupil response algorithm 370. In some examples, the abnormal pupil response algorithm 370 may access a data array structure 358 and evaluate a velocity measurement of the rate of change of the pupil diameter. For example, during the arithmetic task described in relation to FIG. 3, a pupillary dilation response is expected based on the arithmetic problems presented to the user. The dilation may be measured and the velocity of the dilation may be calculated from the video streams. The change in total diameter and velocity of pupil dilation and subsequent pupil constriction can then be compared to an arithmetic threshold to evaluate at 362 the presence of an ocular movement abnormality 364. For example, a change in total diameter can be compared against an expected response from a healthy individual. In testing, healthy individuals show an average change of 19.3 pixels vs. 8.36 pixels in PD patients. In some embodiments, a diameter change threshold may be a diameter change that is less than 70% of the expected change in a healthy individual. In other embodiments, a diameter change threshold may be a diameter change that is less than 60% of the expected change in a healthy individual. In yet other embodiments, a diameter change threshold may be a diameter change that is less than 50% of the expected change in a healthy individual.

The presentation of arithmetic problems to elicit a pupillary response may have benefits in a NED, such as that described herein. In contrast to conventional pupillary dilation tests using arithmetic tasks, lighting in a NED can be explicitly controlled, eliminating the need for complex calculations accounting for pupillary changes due to environmental light. In testing of the present system, the arithmetic tasks were able to generate a pupil response in less than 20 seconds. Average pupil dilation amplitude for all arithmetic tasks was 136.37 pixels vs. 123.9 pixels for static tasks (including saccade). A paired t-test revealed a significant effect of task on pupil size (tₛₜₐₜ=−4.42, P<0.001). The short duration with which a system according to the present disclosure can evoke a pupillary response may be beneficial to physicians and researchers studying cognition for tasks with a short duration.

In further embodiments, the abnormality detection algorithms 326 may include an oscillation/ocular tremor detection algorithm 372. Ocular tremor, or small amplitude oscillation, has been shown to occur in patients with Parkinsonism. A framerate of the video streams of at least 20 Hz may record ocular tremors, as this type of tremor has a frequency of between 4 and 7 Hz.

The oscillation/ocular tremor detection algorithm 372 may include a fast-Fourier transform (FFT) to measure the difference in amplitude of the 4-7 Hz frequency components. Alternatively, FFT could be used to identify and output video segments in which tremor may be present. The FFT may identify the presence of oscillation/ocular tremors between 4-7 Hz by comparison against controls before evaluating at 362 the presence of a ocular movement abnormality 364. In some embodiments, the presence of a ocular movement abnormality 364 may be determined by the data exceeding an oscillation/ocular tremor frequency threshold. In some embodiments, the oscillation/ocular tremor frequency threshold may be when oscillation/ocular tremors between 4-7 Hz occur in a patient twice as frequent as in a control patient. In other embodiments, the oscillation/ocular tremor frequency threshold may be when oscillation/ocular tremors between 4-7 Hz occur in a patient that are three times as frequent as in a control patient.

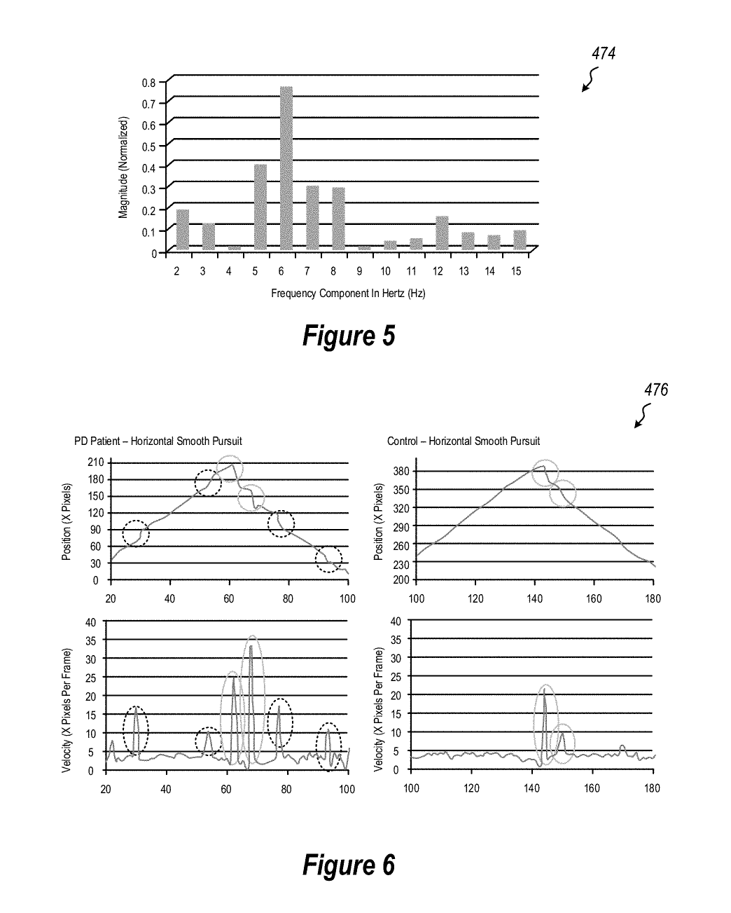

FIG. 5 is a graph 474 illustrating example data of oscillation/ocular tremor from testing of an embodiment of a diagnostic system according to the present disclosure. The FFT analysis represents the difference in frequencies in patients vs. controls of all 32-sample windows in all videos for both groups in the x-direction. Differences are evident in the 5 and 6 Hz range. The average number of frames for which the 5 or 6 Hz components were stronger were greater in PD (44.01%) than controls (34.14%), though it may be more practical to identify particular regions that are likely to contain tremor for remote visual inspection.

FIG. 6 is a series of graphs 476 illustrating example data of abnormal smooth pursuit data with abnormal saccadic intrusions identified by an embodiment of a diagnostic system according to the present disclosure. The left column of graphs illustrates the position and associated velocity of the pupil center of a PD patient, and the right column of graphs illustrates the position and associated velocity of the pupil center of a control patient.

As a user observes a moving target, the movement of the pupil center should be smooth. The peak position at the center of each positional graph (top row) is the point at which the target changes directions, which is followed by one or two normal corrective saccades (circled in solid lines). The regions to the left and right of the central peak should be approximately constant in velocity, as shown in the control velocity graph (bottom right) where the velocity is between 3 and 5 pixels per second except for the expected saccades upon directional change.

In contrast, the PD patient velocity graph (bottom left) indicates there are at least four abnormal saccade intrusions (circled in dashed lines). A velocity threshold of at least 5 pixels per frame greater than expected velocity identified six saccades with two of six being the normal corrective saccades. The remaining four may be identified as potentially abnormal events and either reported as the presence or probability of an abnormality or flagged for subsequent visual review in the video by a medical professional.

Because some of the abnormalities may be identified by the abnormality detection algorithms, but a diagnosis may require further visual review, a visualization module of the system may provide a medical professional with an opportunity to visually inspect the events identified as abnormalities. FIG. 7 illustrates an embodiment of a visualization module 532.

A video input 578 may provide the visualization module with the evaluated video streams from the eye-tracking camera. The visualization module 532 may perform one or more functions on the video data to visually represent the detected abnormalities. The video input 578 may undergo a virtual overlay of the pupil center and contour ellipse, a background subtraction and filtering method (TSUB), Eulerian Video Magnification (EVM), or combinations thereof. For example, the TSUB may include a simple background subtraction with an additional eye-tracking correction. The background subtraction alone may result in a very noisy product, rendering the minute abnormalities difficult to distinguish from other movements. By incorporating a thresholding portion 580, the background subtraction provides only the movement of the high contrast portions of the eye, for example, the edges of the pupil where the pupil meets the iris.

In some embodiments, the visualization module 532 may highlight the pupil region of the recording pupil location 584 and the highlight the pupil region of the expected (e.g., control or calculated) pupil location 586 to visualize displacement. An aggregate frame threshold 588 may allow the system to visualize and highlight the identified abnormalities across multiple frames in addition to the instantaneous evaluation of each frame from the expected values.

One example of aggregate frame analysis is EVM. The application of EVM may amplify motion within the frame of the video input 578 to enhance the detection of movement in the image. In some embodiments, the simultaneous application of EVM and TSUB may allow for detection and measurement of ocular movements in the video that are otherwise imperceptible to an unassisted physician.

The differences between the measured and expected positions in the video may be visualized by colorization 590, line thickening 592 of the measured positions, which may then be merged and overlaid 593 on the original video, such that the displacements appear as highlighted or colored regions on the video. This may display both the presence and degree of displacement from the expected positions. The visualization module may produce the output visualization 594 for review by a medical professional or for storage for later review or archiving.

A diagnostic system according to the present disclosure may collect and capture new and/or more precise data than conventional methodologies. The new data may be compared to the findings of conventional medical evaluations to improve the diagnostic capabilities of the system over time. FIG. 8 illustrates an embodiment of machine learning module 628. For example, the data collected regarding patient interaction with the tasks, regarding eye characteristics, and regarding performance of the tasks may be considered and compared to known, anonymized disease data 696 that is updated and accessed from a remote location, such as a cloud storage device 695.

The diagnostic system may compare the known, anonymized disease data 696 to the measured data through a machine learning, support vector machine, or neural network approach 698 to verify the identification of abnormalities against the known disease data 696 and refine one or more of the threshold values described in relation to the abnormality identification algorithms and refine the identification of abnormalities using new data obtained from the cloud storage device 695. The refinement may allow the diagnostic system to estimate the probability of an abnormality and/or improve a recommendation for diagnosis 699 to a medical professional.

For example, the known, anonymized disease data 696 may include information regarding the data collected regarding patient interaction with the tasks, regarding eye characteristics, and regarding performance of the tasks and may correlate that information with positive and/or negative diagnoses of one or more diseases. Newly collected data may be added to the known, anonymized disease data 696 to further refine the known, anonymized disease data 696 and associated understanding of correlated symptoms and behaviors. The refined known, anonymized disease data 696 may allow for machine learning and further refinement of the threshold values of the abnormality detection algorithms, as well as for further refinement of the probability of an abnormality when collected data is compared against the newly refined known, anonymized disease data 696.

In some embodiments, the iterative refinement of the known, anonymized disease data 696 through machine learning may improve the diagnosis rate of one or more diseases. In other embodiments, periodic review of the known, anonymized disease data 696 by a clinician may avert unintended feedback or iterative loops of the machine learning that may create artifacts in the known, anonymized disease data 696. For example, the machine learning may evaluate only an identified set of values in the collected data, while a pattern may be evident to a clinician evaluating the entirety of the data. The clinician may then add that parameter as a considered parameter that may be compared against collected data to further refine the threshold values of the abnormality detection algorithms.

In at least one embodiment, a diagnostic system according to the present disclosure may provide a target on a NED that moves through a series of tasks to elicit eye movements and pupil changes. The eye movements may be measured by an eye-tracking camera that provides pupil location and diameter information to a processor that is configured to perform one or more abnormality identification algorithms on the pupil location and diameter information to identify and visualize abnormalities in the movement of the patient's eye.

One or more specific embodiments of the present disclosure are described herein. These described embodiments are examples of the presently disclosed techniques. Additionally, in an effort to provide a concise description of these embodiments, not all features of an actual embodiment may be described in the specification. It should be appreciated that in the development of any such actual implementation, as in any engineering or design project, numerous embodiment-specific decisions will be made to achieve the developers' specific goals, such as compliance with system-related and business-related constraints, which may vary from one embodiment to another. Moreover, it should be appreciated that such a development effort might be complex and time consuming, but would nevertheless be a routine undertaking of design, fabrication, and manufacture for those of ordinary skill having the benefit of this disclosure.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

The terms “approximately,” “about,” and “substantially” as used herein represent an amount close to the stated amount that still performs a desired function or achieves a desired result. For example, the terms “approximately,” “about,” and “substantially” may refer to an amount that is within less than 5% of, within less than 1% of, within less than 0.1% of, and within less than 0.01% of a stated amount. Further, it should be understood that any directions or reference frames in the preceding description are merely relative directions or movements. For example, any references to “up” and “down” or “above” or “below” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A system for identifying abnormal eye movements, the system including:
a near-eye display (NED);
an eye-tracking camera;
a frame supporting the NED and the eye-tracking camera; and
a processor in data communication with the NED, the eye-tracking camera, and a computer readable medium having instructions thereon that when provided to the processor cause the processor to:
provide a target on the NED to a user's eye,
change the target or move the target to a plurality of locations on the NED according to one or more tasks of a task module,
record positional information and pupil information of the user's eye during the one or more tasks of the task module, and
compare the positional information and pupil information to at least one threshold value of an abnormality identification algorithm.

2. The system of claim 1, the instructions further including a visualization module that overlays a displacement of the positional information in an output visualization.

3. The system of claim 1, the instructions further including storing at least the positional information and pupil information on a storage device.

4. The system of claim 1, the task module including an instantaneous positional change emulation task.

5. The system of claim 1, the task module including a gradual/oscillatory emulation task.

6. The system of claim 1, the task module including a stationary task.

7. The system of claim 1, the task module including an arithmetic/mathematic task.

8. The system of claim 1, the task module including head coupling wherein movement of the frame is compared to the positional information.

9. The system of claim 1, the eye-tracking camera having an angular resolution of less than 0.1 degrees.

10. A method of identifying abnormal eye movements in a patient, the method comprising:
providing a target on the NED to a user's eye;
moving the target to a plurality of locations in three dimensions on the NED according to one or more tasks of a task module;
recording positional information and pupil information of the user's eye during the one or more tasks of the task module; and
comparing the pupil information to at least one threshold value of an abnormality identification algorithm including at least an abnormal pupil response algorithm.

11. The method of claim 10, the abnormality identification algorithm including a square wave jerk algorithm.

12. The method of claim 11, the square wave jerk algorithm including a velocity threshold value greater than 15 pixels per frame.

13. The method of claim 10, the abnormality identification algorithm including an abnormal smooth pursuit algorithm.

14. The method of claim 13, the abnormal smooth pursuit algorithm including an acceleration threshold value greater than 15 pixels per frame.

15. The method of claim 10, the abnormal pupil response algorithm including a diameter change threshold value that is less than 70% of the expected change in a healthy individual.

16. The method of claim 10, the abnormal pupil response algorithm including a diameter change threshold value greater than 8 pixels.

17. The method of claim 10, the abnormality identification algorithm including an oscillation/ocular tremor detection algorithm.

18. The method of claim 17, the oscillation/ocular tremor detection algorithm including an oscillation/ocular tremor frequency threshold value greater than twice a control value for a quantity of detected ocular oscillation/ocular tremors between 4 and 7 Hz in the positional information.

19. A method of identifying abnormal eye movements in a patient, the method comprising:
providing a target on the NED to a user's eye;
moving the target to a plurality of locations on the NED according to at least a one task of a task module;
recording diagnostic information of the user's eye during the task of the task module;
comparing the diagnostic information to at least one threshold value of an abnormality identification algorithm including at least an abnormal pupil response algorithm;
comparing the diagnostic information against anonymized disease data; and
estimating probability of abnormality based at least partially upon the anonymized disease data.

20. The method of claim 19, further comprising refining the threshold value based upon a comparison of the positional information and pupil information against anonymized disease data.
